(19) United States
(12) Patent Application Publication
(10) Pub. No.: US 2008/0209932 A1
(43) Pub. Date: Sep. 4, 2008

(54) Cooling Device

(76) Inventors: David Conrad Clarke, Christchurch (NZ); Glenn Andrew Clarke, Timaru (NZ)

Correspondence Address: JOHN ALEXANDER GALBREATH, 2516 CHESTNUT WOODS CT, REISTERSTOWN, MD, 21136, US

(21) Appl. No.: 12/088,446

(22) PCT Filed: Sep. 29, 2006
(86) PCT No.: PCT/NZ2006/000252

§ 371 Date: Mar. 28, 2008

(30) Foreign Application Priority Data
Sep. 29, 2005 (NZ) 542067

(51) Int. Cl. F25D 16/00; A61F 7/00; A01K 13/00

(52) U.S. Cl. 62/259.3; 607/108; 119/850

(57) ABSTRACT

A cooling device, which includes a cooling solution and an absorption means; such that the cooling solution includes one or more low boiling point alcohols, one or more cooling sensates and water; wherein the total concentration of low boiling point alcohols is between 5% and 25% of the cooling solution; the total concentration of cooling sensates is up to 20% of the cooling solution, and the concentration of any single cooling sensate is a maximum of 10% of the cooling solution; and the water concentration is at least 50% of the cooling solution, wherein at least part of the absorption means is configured to releasably absorb any cooling solution applied to it and, in use, allow contact between a user's skin and the cooling solution; and wherein said absorption means is a flexible garment configured to closely fit one or more body parts of the user.

### FIELD OF THE INVENTION

This invention relates to the field of localised cooling of an animal to minimise the effect of, or assist with the healing of, soft tissue injuries and improve athletic performance. More particularly the invention relates to cooling bandages and cooling suits. The invention has been developed especially for human beings and thus is described with particular reference to this application. However, the cooling device can also be used with any animal including domestic, zoo and farm animals.

### BACKGROUND OF THE INVENTION

It is well known that for a soft tissue injury, locally cooling the injured area reduces the healing time and pain experienced and thus many coaches and others who deal with people shortly after injury have been taught terms like RICE (Rest Ice Compression Elevation) to remember the recommended initial treatment required. The ice provides localised cooling which combined with the elevation of the injured area and a compression bandage stabilises the injured area, reduces the pain and prevents swelling; all of which aids rapid recovery.

Traditional methods of cooling include ice (either shaved or other forms), frozen vegetables, frozen gel packs and chemical cooling packs. The chemical cooling packs depend on an endothermic chemical reaction to lower their temperature; these can be expensive and are generally less effective than ice/frozen packs.

The ice/frozen parks provide good cooling, if they are able to be shaped to the injured area, but once removed from their refrigerated storage area they start to warm up and their effectiveness drops. For this reason the ice/frozen packs are not generally used for portable applications a long way from refrigeration; instead, the chemical cooling packs are used. Unfortunately this is far from ideal as endothermic chemical cooling packs carry much less cooling potential than the ice/frozen alternatives.

It has further been found that when athletes compete in warm or warm and/or humid environments pre-cooling can assist performance; one study indicates performance improvements between 1% and 4%. To effect this cooling a variety of methods have been tried, the most common being immersion in a bath of cool water, damp towels or water soaked jackets and ice jackets. Chilled air could also be used but at present there are no practical systems available.

The cooling bath is effective at lowering the body's core temperature but it requires a large bath of cool water close to the competition area and a significant immersion time to be effective.

The damp towels or water soaked jackets depend on the evaporation of water from the surface to provide cooling; these require cool water and are less effective than the ice jackets which have become the preferred pre cooling solution.

The ice jackets contain a series of pockets into which ice or frozen gel packs are placed, this has the advantage of allowing the jackets to be portable and as such they are the presently preferred form of pre-cooling. Disadvantages include the requirement for ice or frozen gel packs, and the localised nature of the cooling which limits their ability to lower the core temperature.

There is therefore a need for a portable cooling device that has a high cooling potential, is easy to transport and does not require onsite refrigeration. It would also be useful if the device could be recharged on site with minimal cost or time.

It is therefore an object of the present invention to provide a portable cooling/chilling device that does not require onsite refrigeration and has a cooling capacity greater than that offered by existing cooling packs or devices. A further object of the present invention is to allow the application of compression to an injured area at the same time as cooling. The invention also offers a useful choice to the consumer.

### DISCLOSURE OF THE INVENTION

The present invention provides a cooling device, which includes a cooling solution and an absorption means; such that the cooling solution includes one or more low boiling point alcohols, one or more cooling sensates and water; wherein the total concentration of low boiling point alcohols is between 5% and 25% of the cooling solution; the total concentration of cooling sensates is up to 20% of the cooling solution, and the concentration of any single cooling sensate is a maximum of 10% of the cooling solution; and the water concentration is at least 50% of the cooling solution, wherein at least part of the absorption means is adapted to releasably absorb any cooling solution applied to it and, in use, allow contact between a user's skin and the cooling solution as the cooling solution evaporates; and wherein said absorption means is a flexible garment configured to closely fit one or more body parts of the user.

Preferably the or each low boiling point alcohol is selected from the list consisting of methanol, ethanol, 1-propanol and 2-propanol, and the cooling sensate is a compound that is adapted to quickly provide a cooling sensation. It is further preferred that the or each cooling sensate is a compound such as menthol, an amide, eucalyptus oil, peppermint oil, other volatile essential oils or a manufactured chemical compound with the same properties.

Preferably the cooling solution includes one or more additional components selected from the list consisting of surfactants, colouring agents, arnica, calendula, pain relief agents, anti-inflammatory agents, perfume, odour eliminators, gelling agents, viscosity modifiers, surface tension modifiers and stabilisers.

Preferably said absorption means is flexible, elastic and retains its shape well. In one form the entire absorption means is available to absorb the cooling solution.

Preferably the absorption means is a flat strip of a porous, flexible and elastic material which is adapted to wrap around a portion of the user's body and apply compression to that portion as the cooling solution evaporates.

In a preferred form the absorption means includes one or more absorption sections configured to releasably absorb the cooling solution. In a further preferred form the absorption means includes one or more one or more non-absorbent sections, such that the or each non-absorbent section is configured to prevent the cooling solution from contacting certain areas of the user.

In a highly preferred form the absorption means is a close fitting shirt, such as a tee-shirt. It is preferred the close fitting shirt is adapted to make intimate contact with the user's skin, and has non-absorbent areas such that the cooling solution can only be applied to specific areas. In a further preferred form the close fitting shirt includes areas adapted to apply compression to certain areas of the user's body.

In a further highly preferred form the absorption means is a body suit which includes a first portion and a second portion wherein the first portion is adapted to cover and make intimate contact with the user's arms and torso, and the second portion is adapted to make intimate contact with the legs and at least a portion of the user's feet. It is further preferred that the first and second portions include areas which are non-absorbent such that the cooling solution is prevented from contacting certain areas of the user. It is further preferred that the body suit includes areas that are adapted to absorb more cooling solution thus allow the cooling solution to be applied to specific areas of the user; these areas may also include means to support or compress the user's body to aid injury recovery.

Preferably the non-absorbent areas are rendered non-absorbent by the use of a water repellent compound such as a silicone compound.

Preferably the cooling solution is applied to the absorption means being worn by the user. The or each cooling sensate quickly gives the user a cooling sensation which is followed by physical cooling of the area due to the evaporation of the volatile components of the cooling solution.

By way of example only a preferred embodiment of the present invention will be described with reference to the accompanying drawings in which:—

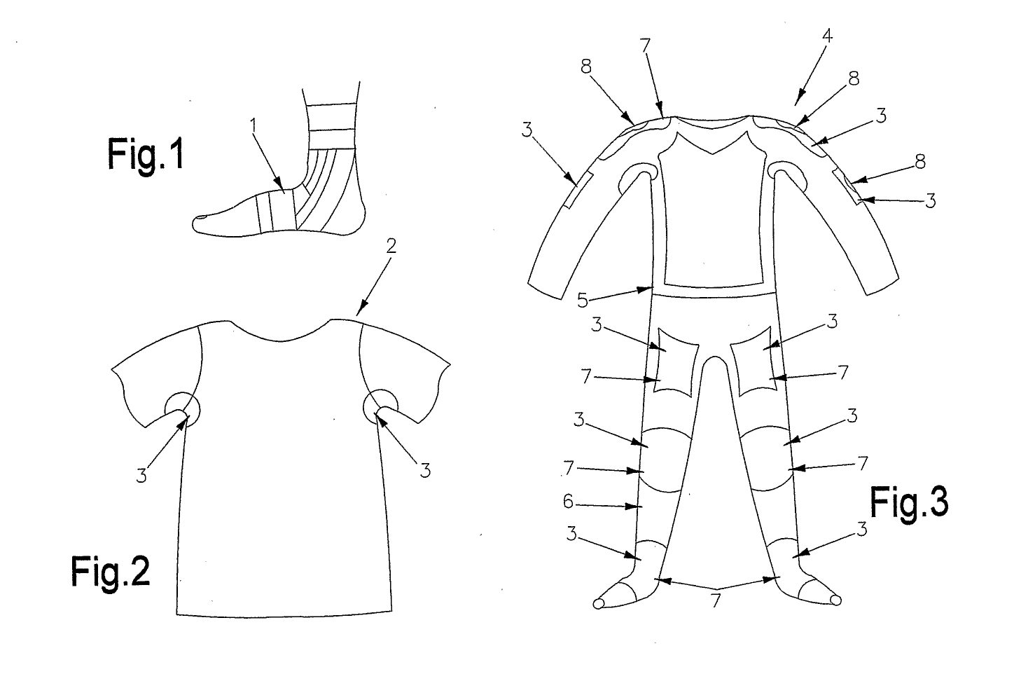

FIG. 1 shows a side view of the bandage applied to an ankle;

FIG. 2 shows a front view of a cooling top; and

FIG. 3 shows a front view of a cooling suit.

With reference to FIG. 1 a cooling bandage (1) is shown wrapped around an ankle (2) the bandage is pre-soaked in a cooling solution. The composition of the cooling solution is as shown in Table 1.

TABLE 1
Cooling Solution Composition

| Concentration | Component | Comment |
|---|---|---|
| 5% to 25% | Low boiling | One or more low |
| | point alcohols | boiling point alcohols |
| Up to 20% | Cooling | One or more cooling |
| | Sensate | sensates, up to 10% of any |
| | | one cooling sensate |
| 50% or more | Water | |
| trace | Modifiers | |

As will be understood by those skilled in the art the cooling solution can also include arnica, calendula or similar materials to aid with recovery or pain relief.

As used herein a low boiling point alcohol is an alcohol such as methanol, ethanol or propanol, i.e. an alcohol that boils at less than about 120° C., and the cooling sensate is one or more selected from amides, menthol, eucalyptus oil, peppermint oil or another essential oil or any chemical compound that generates a sensation of cooling when applied to the skin. The modifiers can modify the smell, viscosity, surface tension, evaporation rate, stability, colour or feel of the cooling solution and to this end they can be such things as perfumes, surfactants and gelling agents.

The cooling solution evaporates from the cooling bandage (1) cooling the bandage (1) and the skin directly, at the same time the cooling sensates give the impression of further cooling. The cooling sensates give the impression of cooling by working on the body's cold receptors, possibly as Menthol does by stimulating entry of Ca²⁺ into the cold receptors thus increasing the intercellular Ca²⁺ concentration in the cold sensitive neurons. These cooling sensates can provide an immediate cooling sensation ahead of the actual cooling effected by the evaporation of the other ingredients, which can reduce the perception of pain. It is well known that physically cooling an injured area while applying compression and elevating the injured area aids in the recovery and results in a stronger and more mobile repair, however it is uncertain as to the reasons for this, though it is believed that the cooling of an injured area, combined with compression and elevation of the injured area, lowers the blood and fluid flow into the injured area which reduces the swelling. Cold also reduces the perception of pain and the occurrence of muscle spasms following an injury which could reduce effects such as shock and secondary damage due to the muscle spasms.

It is possible to recharge the cooling bandage (1) by adding more cooling solution in-situ thus extend the cooling time without disturbing the injured part. The cooling bandage (1) is unlikely to cause frostbite or similar conditions as the evaporative cooling effect will drop as the solution temperature drops thus restricting the minimum temperatures achieved. With the cooling solution directly in contact with the skin the heat transfer from the skin will be higher than that to a cloth covered ice pack, this increased heat transfer will increase the speed of cooling.

Referring to FIG. 2 a cooling top (2) is shown. The cooling top (2) is a close fitting T-shirt shirt made of a flexible porous material which is adapted to absorb said cooling solution (see Table 1 for the cooling solution composition). The cooling top is either pre treated with the cooling solution prior to a user wearing, or the cooling top is put on and the cooling solution is applied. The cooling top (2) is dimensioned to be a snug or tight fit on the user and is adapted to provide good thermal contact with the user such that the cooling solution absorbed by the cooling top.

As the cooling solution contacts the user's skin it quickly creates a cooling sensation, then, as the cooling solution evaporates, it starts to physically cool the user's skin. The cooling top (2) includes protected areas (3) which are pre-treated to prevent them absorbing the cooling solution, these protected areas (3) are adapted to prevent the cooling solution coming into contact with certain areas of the user. These protected areas (3) may be a different material, an applied coating adapted to prevent absorption of the cooling solution or a hole in the cooling top (2).

Referring to FIG. 4 a cooling suit (4) is shown. This cooling suit includes a first part (5) and a second part (6) of a flexible, porous and resilient material. The first part (5) is adapted to cover the torso and arms of a user, and the second part (6) is adapted to cover the legs and part or all of the feet. The construction of the cooling suit (4) is similar to the cooling top (2), thus includes protected areas (3) which are pre-treated to prevent them absorbing the cooling solution, but includes therapeutic areas (7). The therapeutic areas (7) are adapted to provide compression, support and additional cooling to certain areas of the user to aid recovery from an injury or target areas in need of additional cooling or support. These therapeutic areas (7) include cooling areas (8) which are pockets or additional absorbent material attached to the therapeutic areas (7) that are adapted to provide increased or extended localised cooling of the user.

By providing additional support or cooling to specific areas of the user with the therapeutic (7) or cooling (8) areas respectively the user can target injuries or specific muscles/joints that need special attention.

The cooling top (2) and cooling suit (4) can be used for pre-cooling prior to an event or as part of a cooling down regime as well as part of an injury management program.

Though the cooling top (2) and cooling suit (4) are shown adapted for human use they can be shaped for animal use, for example equine cooling suits (4) and bandages (1).

In a further embodiment (not shown) the cooling bandage (1) is a preformed garment configured for specific body parts, thus allowing the cooling of targeted areas without the need for a full body suit. Areas such as the legs of runners, cyclists or horses benefit from targeted cooling after intensive events to prevent lactic acid accumulating in the muscles which can slow recovery.

1. A cooling device, which includes a cooling solution and an absorption means; such that the cooling solution includes one or more low boiling point alcohols, one or more cooling sensates and water; wherein the total concentration of low boiling point alcohols is between 5% and 25% of the cooling solution; the total concentration of cooling sensates is up to 20% of the cooling solution, and the concentration of any single cooling sensate is a maximum of 10% of the cooling solution; and the water concentration is at least 50% of the cooling solution, wherein at least part of the absorption means is configured to releasably absorb any cooling solution applied to it and, in use, allow contact between a user's skin and the cooling solution; and wherein said absorption means is a flexible garment configured to closely fit one or more body parts of the user.

2. The cooling device as claimed in claim 1, characterised in that the or each low boiling point alcohol is selected from the list consisting of methanol, ethanol, 1-propanol and 2-propanol.

3. The cooling device as claimed in claim 1, characterised in that the or each cooling sensate is a compound selected from the list consisting of menthol, an amide, eucalyptus oil, peppermint oil, a volatile essential oil and a manufactured chemical compound that provides a cooling sensation.

4. The cooling device as claimed in claim 1, characterised in that the cooling solution includes one or more additional components selected from the list consisting of surfactants, colouring agents, arnica, calendula, pain relief agents, anti-inflammatory agents, perfume, odour eliminators, gelling agents, viscosity modifiers, surface tension modifiers and stabilisers.

5. The cooling device as claimed in claim 1, characterised in that the absorption means is flexible, elastic and retains its shape well.

6. The cooling device as claimed in claim 1, characterised in that the absorption means includes one or more absorption sections configured to releasably absorb the cooling solution.

7. The cooling device as claimed in claim 1, characterised in that the absorption means includes one or more non-absorbent sections, such that the or each non-absorbent section is configured to prevent the cooling solution from contacting certain areas of the user.

8. The cooling device as claimed in claim 1, characterised in that the garment is configured to apply compression.

9. The cooling device as claimed in claim 1, characterised in that the absorption means is designed to be used by a human being.

10. The cooling device as claimed in claim 1, characterised in that the absorption means is designed to be used by an animal selected from the list consisting of a horse, a greyhound, a farm animal, a zoo animal and a domestic animal.

11. The cooling device as claimed in claim 1, characterised in that the or each body part is independently selected from the list consisting of arm, leg, forearm, upper arm, hand, finger, thumb, shoulder, torso, chest, neck, waist, thigh, calf, knee, hip, abdomen, ankle, foot, toe and fetlock.

12. The cooling device as claimed in claim 1, characterised in that the absorption means is a close fitting shirt.

13. The cooling device as claimed in claim 1, characterised in that the absorption means is a close fitting tee-shirt.

14. The cooling device as claimed in claim 1, characterised in that the absorbtion means is a close fitting shirt adapted to make intimate contact with the user's skin, such that said shirt includes one or more non-absorbent sections, and is adapted to apply compression to certain areas of the users body.

15. The cooling device as claimed in claim 1, characterised in that the absorption means is a body suit which includes a first portion and a second portion wherein the first portion is adapted to cover and make intimate contact with the users arms and torso, and the second portion is adapted to make intimate contact with the legs and at least a portion of the user's feet.

16. The cooling device as claimed in claim 1, characterised in that the absorption means is a body suit which includes a first portion and a second portion wherein the first portion is adapted to cover and make intimate contact with the users arms and torso, and the second portion is adapted to make intimate contact with the legs and at least a portion of the user's feet wherein the first and second portions include one or more first areas which are non-absorbent such that the cooling solution is prevented from contacting certain areas of the user.

17. The cooling device as claimed in claim 15, characterised in that the absorption means is a body suit which includes a first portion and a second portion wherein the first portion is adapted to cover and make intimate contact with the users arms and torso, and the second portion is adapted to make intimate contact with the legs and at least a portion of the users feet such that the body suit includes one or more second areas that are configured to allow more cooling solution to be applied to specific areas of the user.

18. The cooling device as claimed in claim 1, characterised in that the absorption means is a body suit which includes a first portion and a second portion wherein the first portion is adapted to cover and make intimate contact with the users arms and torso, and the second portion is adapted to make intimate contact with the legs and at least a portion of the users feet, wherein the first and second portions include one or more first areas which are non-absorbent and prevent the cooling solution from contacting certain areas of the user said body suit further includes one or more second areas configured to allow more cooling solution to be applied to specific areas of the user, such that the or each first and/or second area includes means to support and/or compress the users body to aid injury recovery.

19. (canceled)

20. The cooling device as claimed in claim 1, characterised in that the absorption means is designed to be used by a human being and said absorption means includes one or more absorption sections configured to releasably absorb the cooling solution.

21. The cooling device as claimed in claim 1, characterised in that the absorption means is designed to be used by an animal selected from the list consisting of a horse, a greyhound, a farm animal, a zoo animal and a domestic animal; and said absorption means includes one or more absorption sections configured to releasably absorb the cooling solution.
